(19) United States
(12) Patent Application Publication
(10) Pub. No.: US 2026/0069820 A1
(43) Pub. Date: Mar. 12, 2026

(54) HEADBOARD FOR EMITTING SLEEP-PROMOTING STIMULI

(71) Applicants: Felicia Kelley-Murray, San Antonio, TX (US); Robert Murray, San Antonio, TX (US)

(72) Inventors: Felicia Kelley-Murray, San Antonio, TX (US); Robert Murray, San Antonio, TX (US)

(21) Appl. No.: 18/882,971

(22) Filed: Sep. 12, 2024

(51) Int. Cl. A61M 21/02; A47C 19/02; A47C 21/00; A61M 21/00

(52) U.S. Cl. CPC A61M 21/02; A47C 21/003; A47C 19/022; A61M 2021/0016; A61M 2021/0027; A61M 2021/0044; A61M 2205/3375; A61M 2205/3561; A61M 2230/63

(57) ABSTRACT

A headboard apparatus for providing various calming stimuli for promoting sleep includes a body which defines a pair of recesses in a front side thereof which are laterally spaced from each other with respect to the body. A pair of doors is mounted to the body, and each door is movably mounted over a respective recess of the pair of recesses. A pair of speakers is mounted to the body, wherein each speaker of the pair of speakers is positioned in a respective recess of the pair of recesses of the body. Each door is selectively positionable over an associated one of the speakers.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to headboards and more particularly pertains to a new headboard for providing various calming stimuli for promoting sleep.

### (2) Description of Related Art including information disclosed under 37 CFR 1.97 and 1.98.

The prior art describes headboards which emit sound or light to promote sleep. However, the prior art fails to describe such an apparatus which includes speakers housed in recesses which may be selectively covered by doors. The prior art also fails to describe such an apparatus in which light sources are positioned in the recesses and in which an aroma diffuser is mounted to the headboard. A central processor may control the speakers, light sources, and aroma diffuser via user input or parameters measured by one or more sensors.

### (h) BRIEF SUMMARY OF THE INVENTION

An embodiment of the disclosure meets the needs presented above by generally comprising a body which defines a pair of recesses in a front side thereof which are laterally spaced from each other with respect to the body. A pair of doors is mounted to the body, and each door is movably mounted over a respective recess of the pair of recesses. A pair of speakers is mounted to the body, wherein each speaker of the pair of speakers is positioned in a respective recess of the pair of recesses of the body. Each door is selectively positionable over an associated one of the speakers.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a headboard apparatus according to an embodiment of the disclosure.

FIG. 2 is an exploded perspective view of an embodiment of the disclosure.

FIG. 3 is a front view of an embodiment of the disclosure.

FIG. 4 is a side view of an embodiment of the disclosure.

FIG. 5 is a detail view of a remote controller of an embodiment of the disclosure.

FIG. 6 is an in-use view of an embodiment of the disclosure.

FIG. 7 is a block diagram of an embodiment of the disclosure.

### (j) DETAILED DESCRIPTION OF THE INVENTION

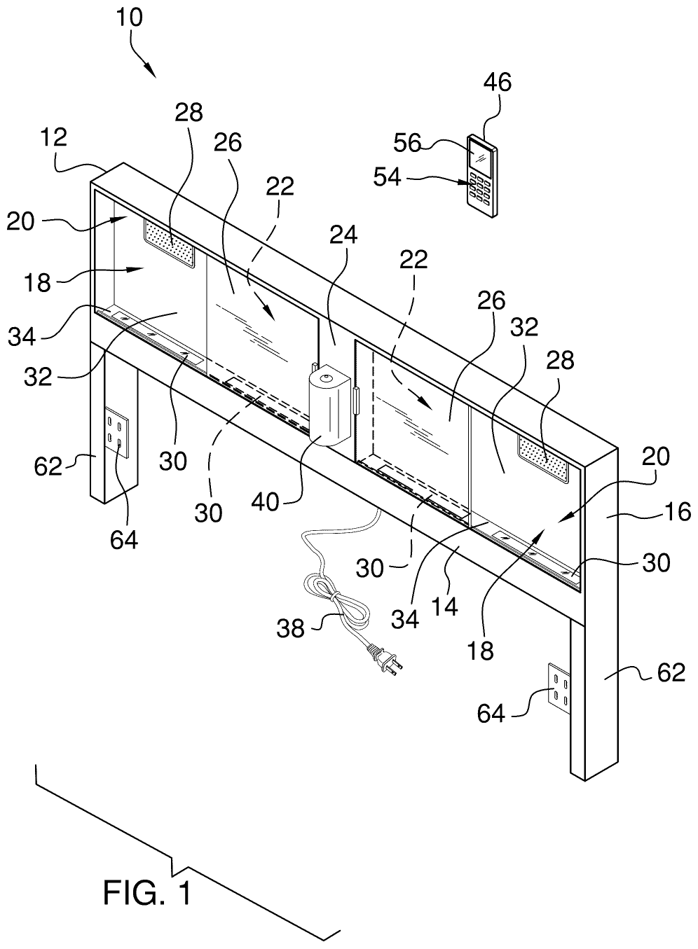

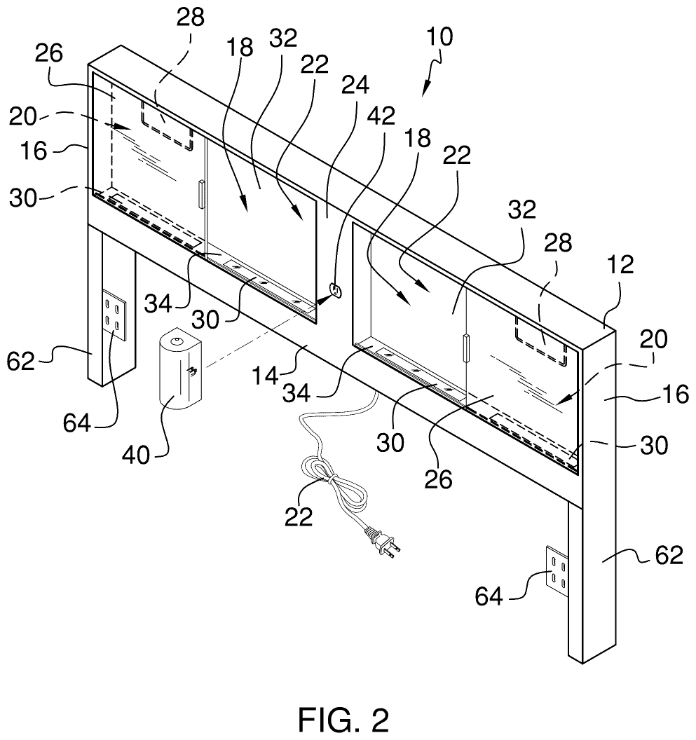

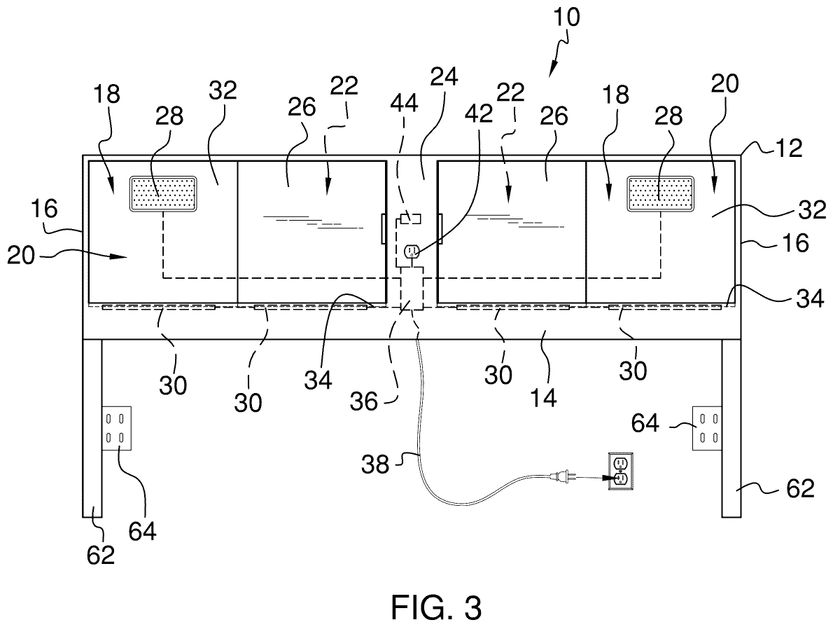

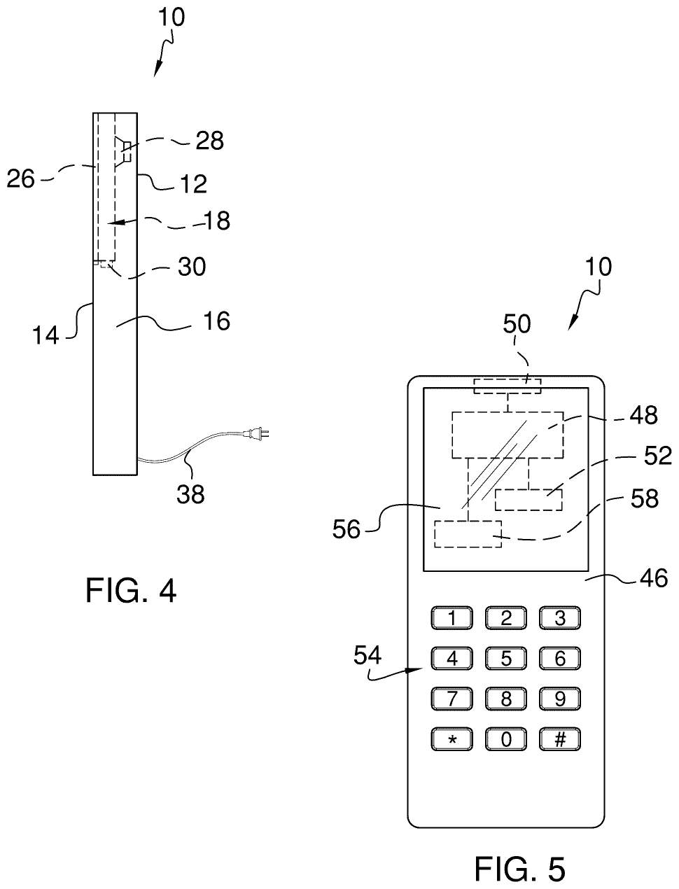

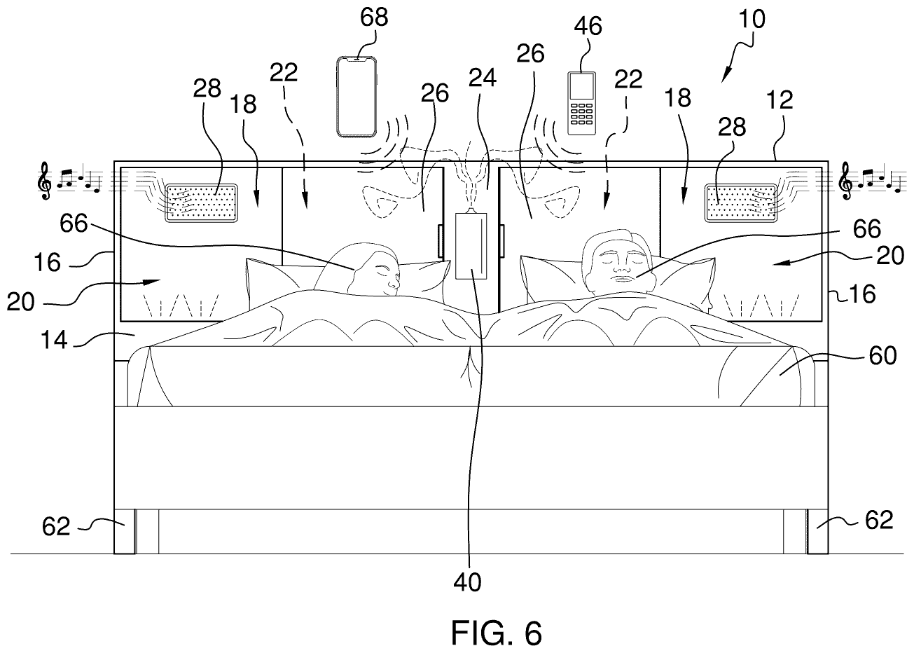

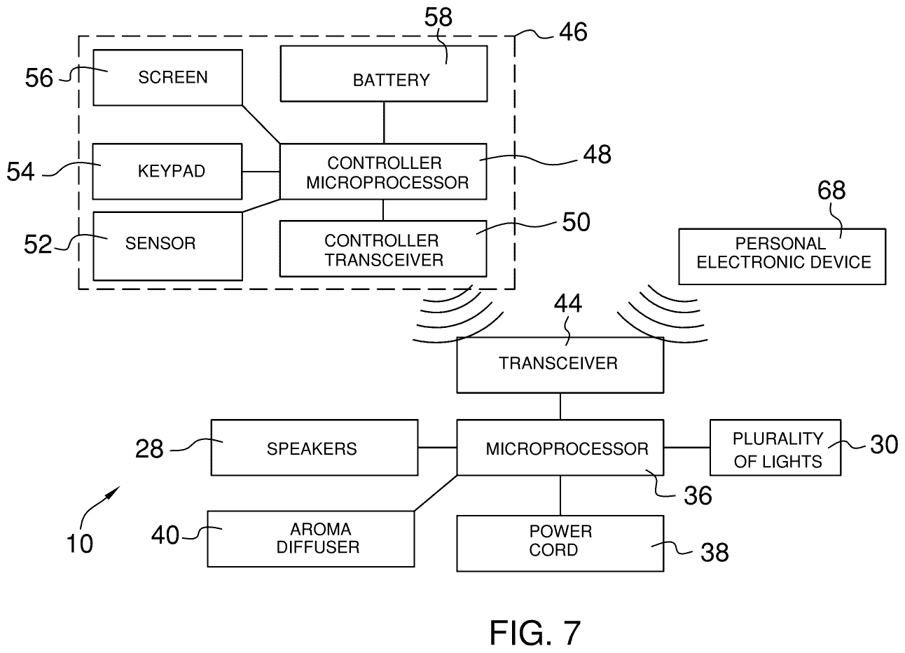

With reference now to the drawings, and in particular to FIGS. 1 through 7 thereof, a new headboard embodying the principles and concepts of an embodiment of the disclosure and generally designated by the reference numeral 10 will be described.

As best illustrated in FIGS. 1 through 7, the headboard apparatus 10 generally comprises a body 12 which is sized and shaped to be placed at a head of a mattress 60 and extend laterally across the mattress 60. A pair of posts 62 are coupled to and extend downwardly from the body 12. Each post 62 has a bracket 64 attached thereto which facilitates attachment of the respective post 62 to a side rail of a bed frame via threaded fasteners. In some embodiments, the body 12, the posts 62, and the side rails may be integrally formed. Varying numbers of posts 62 may be used in various embodiments and various fasteners may be used to attach the body 12 to the side rails. In some embodiments, the body 12 may be mountable to a wall and entirely separate from a bed frame which supports the mattress 60.

The body 12 defines a pair of recesses 18 in a front side 14 thereof. The recesses 18 are laterally spaced from each other with respect to the body 12. Each recess 18 has an outer portion 20 and an inner portion 22, where the outer portion 20 of each recess 18 of the pair of recesses 18 is positioned adjacent to a respective lateral side 16 of a pair of lateral sides 16 of the body 12 and the inner portion 22 of each recess 18 of the pair of recesses 18 is positioned adjacent to a central section 24 of the body 12. The central section 24 is positioned centrally between the pair of lateral sides 16 of the body 12.

A pair of doors 26 is mounted to the body 12. Each door 26 is slidably mounted over a respective recess 18 of the pair of recesses 18. Each door 26 is movable to alternately cover the outer portion 20 and the inner portion 22 of the respective recess 18. In some embodiments, each recess 18 has a pair of doors which are slidably mounted to alternately cover the outer portion 20 and the inner portion 22 thereof. In such embodiments, the doors corresponding to each recess 18 may be slidable along different tracks or grooves from each other such that the entirety of the associated recess 18 may be covered by the pair of doors and one of the doors may be slid to open either the outer portion 20 or the inner portion 22. In some embodiments, the doors may be pivotably mounted, removably mounted, or otherwise movable to selectively cover at least one of the outer portion 20 and the inner portion 22 of each recess 18.

A pair of speakers 28 is mounted to the body 12. Each speaker 28 is positioned in a respective recess 18 of the pair of recesses 18 of the body 12 on a rear interior surface 32 and in the outer portion 20 of the respective recess 18. A plurality of light sources 30 is mounted to the body 12. Each light source 30 is positioned in an associated recess 18 of the pair of recesses 18 of the body 12 on a lower interior surface 34 of the associated recess 18. The outer portion 20 of each recess 18 has one light source 30 positioned therein, and the inner portion 22 of each recess 18 has another one light source 30 positioned therein. A processor 36 is operatively coupled to the pair of speakers 28 and the plurality of light sources 30. The processor 36 is programmed to play audio via the speakers 28 and to selectively activate the light sources 30. A power cord 38 is electrically coupled to the processor 36 and may be coupled to an electrical power source to power the processor 36.

An aroma diffuser 40 is mounted to the central portion of the body 12 between the pair of recesses 18 via an electrical outlet 42 which electrically couples the aroma diffuser 40 to the processor 36. The aroma diffuser 40 may be removed from the electrical outlet 42 to detach the aroma diffuser 40 from the body 12. The processor 36 is programmed to selectively operate the aroma diffuser 40 via selectively powering the electrical outlet 42. In other embodiments, other electrical connections maybe present for transmitting operational signals from the processor 36 to the aroma diffuser 40 to alter a rate of aroma diffusion or other parameters associated with operation of the aroma diffuser 40. The aroma diffuser 40 may be of any suitable type of aroma lamp. For example, the aroma diffuser 40 may be a heat diffuser type which heats scented oils or similar substances to diffuse them into the air or a nebulizer type which forms a scented solution or suspension into an aerosol via ultrasonic vibration or similar stimulus.

A transceiver 44 is operatively coupled to the processor 36 which facilitates control of the processor 36 via wireless communication with a remote controller 46 or a remote personal device 68 such as a smartphone, a tablet, a computer, or the like. The remote controller 46 comprises a controller processor 48 and a controller transceiver 50 which are operatively coupled together. The controller processor 48 is programmed to send control signals to the processor 36 via the controller transceiver 50 and the transceiver 44.

The remote controller 46 comprises a sensor 52 configured for detecting sleep parameters of a user or users 66. For example, the sensor 52 may be an accelerometer which may measure movement of the users 66 during a sleep period by being placed on the bed with the users 66. The sensor 52 may be a microphone which measures snoring and other sounds which may disturb the sleep of the users 66. Multiple sensors of various types may be used to determine one or more sleep parameters. The controller processor 48 may then be programmed to transmit commands to the processor 36 based on the sleep parameters of the users 66. For example, a stimulus known or believed to facilitate re-entry into sleep for the users 66 may be triggered by the processor 36 after the controller processor 48 determines via the sensor 52 that the users 66 has awoken. Likewise, the remote personal device 68 may have programming which detects the sleep parameters via an accelerometer, a camera, a microphone, or another sensor of the remote personal device 68, and the remote personal device 68 may be programmed to send command signals to the processor 36 via the transceiver 44 based upon the detected sleep parameters.

The remote controller 46 includes an interface which includes a keypad 54 for inputting commands and a screen 56 for displaying menu options to the users 66. Other input devices may be used such as a touchscreen, dials, switches, and the like. The controller processor 48 may be powered by a battery 58, the external power source, or any other suitable power supply.

In use, the users 66 may operate the headboard apparatus 10 to play audio such as music, nature sounds, a narrated story, or the like. The light sources 30 may also be selectively activated, and the doors 26 mounted over the recesses 18 of the body 12 may be adjusted to permit a desired amount of audio and light to be heard by the users 66. The aroma diffuser 40 may also be operated as desired via the processor 36. Commands may be sent to the processor 36 via the remote personal device 68 or the remote controller 46. Audio played on the speakers 28 may be stored on the processor 36, the remote controller 46, the remote personal device 68, or on an external memory from which it is streamed or downloaded.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A headboard apparatus comprising:
a body, the body defining a pair of recesses in a front side thereof, the pair of recesses being laterally spaced from each other with respect to the body;
a pair of doors mounted to the body, each door of the pair of doors being movably mounted over a respective recess of the pair of recesses; and
a pair of speakers mounted to the body, each speaker of the pair of speakers being positioned in a respective recess of the pair of recesses of the body, the pair of doors being selectively positionable over the pair of speakers.

2. The headboard apparatus of claim 1, further comprising a processor operatively coupled to the pair of speakers, the processor being programmed to play audio via at least one speaker of the pair of speakers.

3. The headboard apparatus of claim 2, further comprising a plurality of light sources mounted to the body and operatively coupled to the processor, each light source of the plurality of light sources being positioned in an associated recess of the pair of recesses of the body.

4. The headboard apparatus of claim 3, wherein each light source of the plurality of light sources being positioned on a lower interior surface of the associated recess.

5. The headboard apparatus of claim 2, further comprising an aroma diffuser mounted to the body and operatively coupled to the processor.

6. The headboard apparatus of claim 5, wherein the aroma diffuser is positioned on the front side of the body between the pair of recesses.

7. The headboard apparatus of claim 6, wherein the aroma diffuser is removable from the body.

8. The headboard apparatus of claim 7, further comprising an electrical outlet mounted to the body, the electrical outlet mounting the aroma diffuser to the body and electrically coupling the aroma diffuser to the processor.

9. The headboard apparatus of claim 2, further comprising a transceiver operatively coupled to the processor.

10. The headboard apparatus of claim 9, wherein the processor is configured to be in wireless communication with a remote personal device via the transceiver.

11. The headboard apparatus of claim 9, further comprising a remote controller in wireless communication with the processor via the transceiver, the processor being programmed to be controlled by commands received by the remote controller.

12. The headboard apparatus of claim 11, wherein the remote controller comprises a sensor configured for detecting sleep parameters of a user, the remote controller being configured to transmit commands to the processor based on the sleep parameters of the user.

13. The headboard apparatus of claim 1, wherein:
each recess of the pair of recesses having an outer portion and an inner portion, the outer portion of each recess of the pair of recesses being positioned adjacent to a respective lateral side of a pair of lateral sides of the body, the inner portion of each recess of the pair of recesses being positioned adjacent to a central section of the body which is positioned centrally between the pair of lateral sides of the body;
each door being movable to alternately cover the outer portion and the inner portion of the respective recess, each door of the pair of doors being slidably mounted to the body; and
each speaker of the pair of speakers being positioned on a rear interior surface of the respective recess, each speaker of the pair of speakers being positioned in the outer portion of the respective recess.

14. The headboard apparatus of claim 13, further comprising a plurality of light sources mounted to the body and operatively coupled to the processor, each light source of the plurality of light sources being positioned in an associated recess of the pair of recesses of the body, each light source of the plurality of light sources being positioned on a lower interior surface of the associated recess, the outer portion of each recess having one light source of the plurality of light sources positioned therein, the inner portion of each recess having another one light source of the plurality of light sources positioned therein.

15. A headboard apparatus comprising:
a body, the body defining a pair of recesses in a front side thereof, the pair of recesses being laterally spaced from each other with respect to the body, each recess of the pair of recesses having an outer portion and an inner portion, the outer portion of each recess of the pair of recesses being positioned adjacent to a respective lateral side of a pair of lateral sides of the body, the inner portion of each recess of the pair of recesses being positioned adjacent to a central section of the body which is positioned centrally between the pair of lateral sides of the body;
a pair of doors mounted to the body, each door of the pair of doors being movably mounted over a respective recess of the pair of recesses, each door being movable to alternately cover the outer portion and the inner portion of the respective recess, each door of the pair of doors being slidably mounted to the body;
a pair of speakers mounted to the body, each speaker of the pair of speakers being positioned in a respective recess of the pair of recesses of the body, each speaker of the pair of speakers being positioned on a rear interior surface of the respective recess, each speaker of the pair of speakers being positioned in the outer portion of the respective recess;
a processor operatively coupled to the pair of speakers, the processor being programmed to play audio via at least one speaker of the pair of speakers;
a plurality of light sources mounted to the body and operatively coupled to the processor, each light source of the plurality of light sources being positioned in an associated recess of the pair of recesses of the body, each light source of the plurality of light sources being positioned on a lower interior surface of the associated recess, the outer portion of each recess having one light source of the plurality of light sources positioned therein, the inner portion of each recess having another one light source of the plurality of light sources positioned therein;
an aroma diffuser mounted to the body and operatively coupled to the processor, the aroma diffuser being positioned on the front side of the body between the pair of recesses, the aroma diffuser being removable from the body;
an electrical outlet mounted to the body, the electrical outlet mounting the aroma diffuser to the body and electrically coupling the aroma diffuser to the processor;
a transceiver operatively coupled to the processor, the processor being configured to be in wireless communication with a remote personal device via the transceiver; and
a remote controller in wireless communication with the processor via the transceiver, the processor being programmed to be controlled by commands received by the remote controller, the remote controller comprising a sensor configured for detecting sleep parameters of a user, the remote controller being configured to transmit commands to the processor based on the sleep parameters of the user.
